(19) United States
(12) Patent Application Publication
(10) Pub. No.: US 2022/0054467 A1
(43) Pub. Date: Feb. 24, 2022

(54) TREATMENT OF PSORIASIS WITH TOPICAL TAPINAROF-TAZAROTENE COMBINATION COMPOSITIONS

(71) Applicant: Sol-Gel Technologies Ltd., Ness Ziona (IL)

(72) Inventor: Moshe ARKIN, Kfar Shmaryahu (IL)

(73) Assignee: Sol-Gel Technologies Ltd., Ness Ziona (IL)

(21) Appl. No.: 17/432,927

(22) PCT Filed: Feb. 25, 2020
(86) PCT No.: PCT/IL2020/050211

§ 371 Date: Aug. 22, 2021

Related U.S. Application Data
(60) Provisional application No. 62/809,929, filed on Feb. 25, 2019.

(51) Int. Cl. A61K 31/4436; A61K 31/05; A61K 9/00; A61K 9/06; A61K 47/44; A61K 47/10; A61K 47/20; A61K 47/18; A61K 47/32; A61K 47/12; A61K 47/02; A61K 45/06

(52) U.S. Cl. CPC A61K 31/4436; A61K 31/05; A61K 9/0014; A61K 9/06; A61K 47/44; A61K 45/06; A61K 47/20; A61K 47/183; A61K 47/32; A61K 47/12; A61K 47/02; A61K 47/10

(57) ABSTRACT

Provided herein is a topical combination composition comprising from about 0.25% w/w to about 2.0% w/w tapinarof, from about 0.025% w/w to about 0.1% w/w tazarotene and a carrier suitable for topical administration. Also provided is a topical combination composition comprising from about 0.25% w/w to about 2.0% w/w tapinarof, from about 0.025% w/w to about 0.1% w/w w tazarotene, from about 0.01% w/w to about 0.25% w/w at least one corticosteroid of potency class 1-4 and a carrier suitable for topical administration. The above compositions are useful for the treatment, prevention or alleviation of plaque psoriasis and exhibit synergistic and/or additive effects which allow reducing the amounts of the active agents in the compositions. The addition of tazarotene to tapinarof potentiates the tapinarof anti-psoriatic therapeutic effect.

### FIELD OF THE INVENTION

The present invention, in some embodiments thereof, relates to treatment of psoriasis by topical administration of a combination composition comprising tapinarof and tazarotene. The compositions of this invention are useful for the treatment, prevention or amelioration of psoriasis and exhibit synergistic and/or additive effects which allow reducing the dose of the active agents in the combination composition.

### BACKGROUND

Tapinarof (3,5-dihydroxy-4-isopropyl-trans-stilbene) is a pharmaceutical active agent investigated for the treatment of atopic dermatitis, psoriasis and psoriatic disorders (Zang Y N, et al., Int J Clin Pharmacol Ther. 2016 February; 54(2):87-95). The 3,5-dihydroxy-4-isopropyl-stilbene is also known as benvitimod.

Tapinarof is a first-in-class drug, whose mechanism is not yet fully understood.

Psoriasis is an autoimmune disease, characterized by typically red, scaly patches of skin. There are several types of psoriasis: plaque, guttate, inverse/flexural, pustular, erythrodermic, sebo-psoriasis/seborrheic-like psoriasis, nail psoriasis, palmoplanar psoriasis or scalp psoriasis. Plaque psoriasis (psoriasis vulgaris) is the most common form of psoriasis. An estimated 80 to 90 percent of people with psoriasis have plaque psoriasis. It's characterized by thick red patches of skin, often with a silver or white scaly layer. Guttate psoriasis appears in small red spots on the skin. It's the second most common type, affecting around 8 percent of people with psoriasis. Most of the time it starts during childhood or young adulthood. The spots are small, separate, and drop-shaped. They often appear on the torso and limbs, but they can also appear on your face and scalp. Spots are usually not as thick as plaque psoriasis, but they can develop into plaque psoriasis over time. Inverse psoriasis is a rare form of psoriasis which is also known as flexural or intertriginous psoriasis. This subtype of psoriasis can occur in any area where two skin surfaces meet. Classically the skin of the groin region, armpits and genitals are affected. In these regions the skin appears red, shiny, and moist, with clear borders, and can sometimes crack in the centre. This rare form of psoriasis accounts for 3-7% of people with psoriasis. A small Chinese study found that the average age of onset for inverse psoriasis is 28.9 years. Occasionally people with another subtype of psoriasis known as pustular psoriasis go on to develop inverse psoriasis. Recent guidelines from the National Psoriasis Foundation recommend the use of low to moderate strength corticosteroids for flare ups of this type of psoriasis and calcipotriene and either tacrolimus or pimecrolimus (e.g. Elidel) for treatment of inverse psoriasis in the long term. Pustular psoriasis is a severe form of psoriasis. It develops fast in the form of many white pustules surrounded by red skin. Pustular psoriasis may affect isolated areas of the body, like the hands and feet, or cover most of the skin's surface. These pustules can also join together and form scaling. Erythrodermic psoriasis, or exfoliative psoriasis, is a rare psoriasis type that looks like severe burns. The condition is serious, and can be a medical emergency. This form of psoriasis is widespread, red, and scaly. It may cover large portions of the body. Exfoliation often occurs in larger pieces than the small scales typical to most psoriasis. Sebo-psoriasis/Seborrheic-like psoriasis is a common form of psoriasis with clinical aspects of psoriasis and seborrheic dermatitis, and it may be difficult to distinguish from the latter. This form of psoriasis typically manifests as red plaques with greasy scales in areas of higher sebum production such as the scalp, forehead, skin folds next to the nose, skin surrounding the mouth, skin on the chest above the sternum, and in skin folds. Nail Psoriasis: psoriasis can affect the nails and produces a variety of changes in the appearance of finger and toe nails. Nail psoriasis occurs in 40-45% of people with psoriasis affecting the skin and has a lifetime incidence of 80-90% in those with psoriatic arthritis. These changes include pitting of the nails (pinhead-sized depressions in the nail is seen in 70% with nail psoriasis), whitening of the nail, small areas of bleeding from capillaries under the nail, yellow-reddish discoloration of the nails known as the oil drop or salmon spot, dryness, thickening of the skin under the nail (subungual hyperkeratosis), loosening and separation of the nail (onycholysis), and crumbling of the nail. Palmoplantar Psoriasis is a chronic autoimmune disease characterized by the rise of desquamative plaques on the palms and soles. Due to the thick stratum corneum of the palmoplantar regions, the search for effective topical treatments has been significantly more difficult than other forms of psoriasis. Current topical treatments include phototherapy, methotrexate gel, laser therapy, and tazarotene ointment; most treatments outside of phototherapy, however, do not have sufficient high-level clinical evaluations to justify their efficacy. In this systematic review, we explore the literature on different topical treatment regimens for palmoplantar psoriasis. Scalp Psoriasis is a common skin disorder that makes raised, reddish, often scaly patches. It can pop up as a single patch or several, and can even affect your entire scalp. It can also spread to your forehead, the back of your neck, or behind and inside your ears. About half of the estimated 7.5 million Americans with psoriasis—which can affect any skin surface—have it on their scalp. Sometimes the scalp is the only place they have it, but that's uncommon. Scalp psoriasis can be mild and almost unnoticeable. But it can also be severe, last a long time, and cause thick, crusted sores. Intense itching can affect your sleep and everyday life, and scratching a lot can lead to skin infections and hair loss.

Though a number of psoriasis treatments are available, most treatments bring about symptom alleviation or remission rather than complete cure.

### SUMMARY OF THE INVENTION

This invention provides a topical combination composition comprising from about 0.25% w/w to about 2.0% w/w tapinarof, from about 0.025% w/w to about 0.1% w/w tazarotene and a carrier suitable for topical administration.

Also provided is a topical combination composition comprising from about 0.25% w/w to about 2.0% w/w tapinarof, from about 0.025% w/w to about 0.1% w/w tazarotene, from about 0.01% w/w to about 0.25% w/w at least one corticosteroid of potency class 1-4 and a carrier suitable for topical administration.

The compositions are suitable for the treatment, prevention or alleviation of plaque psoriasis and exhibit synergistic and/or additive effects which allow reducing the amounts of the active agents in the compositions. The addition of tazarotene to tapinarof potentiates the tapinarof anti-psoriatic therapeutic effect.

### DETAILED DESCRIPTION OF THE INVENTION

The present invention provides novel methods of treatment and topical compositions useful for the treatment, prevention and amelioration of psoriasis.

In another embodiment, the psoriasis is plaque psoriasis, guttate psoriasis, inverse/flexural psoriasis, pustular psoriasis, erythrodermic psoriasis, sebo-psoriasis/seborrheic-like psoriasis, nail psoriasis, palmoplanar psoriasis or scalp psoriasis. In another embodiment, the psoriasis is plaque psoriasis. In another embodiment, the psoriasis is inverse/flexural psoriasis.

Some of the known topical psoriasis treatments use pharmaceutical active agents selected from corticosteroids like halobetasol or desoxymethasone and Vitamin D analogues like calcipotriene or paricalcitol. Combinations of several of the above classes of active agents, like Vitamin D and corticosteroids have been investigated. Most of the known psoriasis topical treatments in general, and those comprising steroids in particular, present undesirable side-effects.

Tapinarof

Tapinarof (3,5-dihydroxy-4-isopropyl-trans-stilbene, benvitimod, GSK2894512) is a first-in-class drug, whose mechanism is not yet fully understood. It is being developed by Glaxo Smith Kline (Stiefel, a GSK company) and Dermavant as a topical drug for treatment of mild to moderate plaque psoriasis and atopic dermatitis. It was shown in both mouse models and in vitro human skin studies to inhibit specific proinflammatory mediators, including interleukin-6 and interleukin-17A, and enhance skin barrier function (J Invest Dermatol. 2017 October; 137[10]:2110-9).

A number of GSK28994512 (tapinarof) clinical studies were completed:
1. Dose finding study (Phase I) of GSK28994512 in subjects with plaque psoriasis—1% (10 mg/g) and 0.5% (5 mg/g) creams for two application frequencies, once a day and twice a day, vs. vehicle cream.
2. Dose finding study of GSK28994512 (Phase II) in subjects with atopic dermatitis—1% (10 mg/g) and 0.5% (5 mg/g) creams for two application frequencies, once a day and twice a day, vs. vehicle cream.
3. Dose finding study (Phase II) of GSK28994512 in subjects with plaque psoriasis—1% (10 mg/g) and 0.5% (5 mg/g) creams for two application frequencies, once a day and twice a day, vs. vehicle cream.
4. Skin irritation study (Phase I) of GSK28994512 cream (0.5% and 1%) vs vehicle cream.
5. A single dose (Phase I) exploratory study in healthy volunteers with GSK28994512 cream for atopic dermatitis. Two cream formulations (GSK2894512 cream A and GSK2894512 cream B) were tested in combination for skin residency.
6. Pharmacokinetic study (Phase I) of topical GSK2894512 1% and 2% creams following twice daily application of the 2% cream (cohort 1) or 1% cream (cohort 2).

According to Jancin B. (Dermatology News, Nov. 11, 2017), in the above studies, the 1% tapinarof arm showed higher efficacy and had a quicker onset of action than the 0.5% arm or vehicle. The most frequent adverse events associated with tapinarof were folliculitis and contact dermatitis. Tapinarof, which seems to be a significant advance in psoriasis treatment, presents however higher adverse effects (44.5%) when compared to placebo (20.2%) and calcipotriene (19.5%) as a single drug (Medscape Mar. 5, 2017 report by Frellick M., on Abstr. 5629, Amer. Acad. of Dermatology meeting, Mar. 4, 2017).

Tazarotene

Tazarotene is a retinoid, a class of chemical compounds structurally related to Vitamin A which are effective in the treatment of a number of skin disorders, like acne and psoriasis. Tazarotene (marketed as Tazorac, Avage, Zorac, and Fabior) is a third-generation prescription topical retinoid sold as a cream, gel, or foam. It is commonly sold in two concentrations: 0.05% and 0.1%. Tazorac® (tazarotene) 0.05% and 0.1% topical gel is FDA-approved for the treatment of plaque psoriasis of up to 20% body surface involvement.

Ortho Dermatologics (division of Bausch Health Companies) has resubmitted to FDA the NDA of DUOBRII™ (IDP-118), a lotion combination product of halobetasol propionate and tazarotene for the treatment of plaque psoriasis.

While DUOBRII™ seems to be a promising product, it has a built-in drawback: halobetasol, one of the two DUOBRII™ active agents is a super-potent corticosteroid and, as such, the combination product can be administered only for limited periods of time, in order to avoid steroid side-effects. Treatment for extended periods of time is important, for example for the therapy of chronic plaque psoriasis cases, and the composition of this invention fills this need.

There is an unmet need for methods for the treatment of plaque psoriasis using tapinarof topical combination compositions, devoid of serious side-effects, which may be used for extended periods of time.

It occurred to the present inventor, that topical combination compositions comprising tapinarof and tazarotene, essentially free of corticosteroids, may allow treatment for longer periods of time, exhibit improved therapeutic effects and also synergistic or additive effects in the treatment of plaque psoriasis and, as a result, allow using lower dosage of the actives and diminish the product's side-effects (like local irritation and contact dermatitis).

In some embodiments, there is provided a topical composition comprising from about 0.25% w/w to about 2.0% w/w tapinarof, from about 0.025% w/w to about 0.1% w/w tazarotene and a carrier suitable for topical administration.

The tapinarof combination composition of this invention has a double advantage vs. the use of tapinarof or tazarotene as single drugs: on the one hand the tapinarof has the potential to alleviate the tazarotene side-effects and on the other hand the synergistic or additive effect may enable using lower active agent amounts. The addition of tazarotene to tapinarof potentiates the tapinarof anti-psoriatic therapeutic effect.

Corticosteroids

Optionally, the above tapinarof-tazarotene composition may further comprise a low corticosteroid amount of from about 0.01% w/w to about 0.25% w/w at least one corticosteroid of potency class 1-4 (see below a list of steroids classified by their potencies), approved and marketed in the US for topical use.

In some embodiments, there is provided a topical composition comprising from about 0.25% w/w to about 2.0% w/w tapinarof, from about 0.025% w/w to about 0.1% w/w tazarotene, from about 0.01% w/w to about 0.25% w/w at least one corticosteroid of potency class 1-4 and a carrier suitable for topical administration.

Topical Steroid Classification by Potency

According to the “Topical steroid potency chart” of the National Psoriasis Foundation (NPF), the various marketed topical drugs comprising steroids belong to the following potency classes, according to the steroid and the topical drug strength. Due to the different topical drug strength, drugs of different strengths and/or different dosage forms may belong to more than one steroid class. The percentages in parentheses are the steroid strengths for the FDA-approved topical steroid compositions.

Class 1—superpotent, comprising 7 steroids: clobetasol propionate (0.05%), flurandrenolide (0.05%), betamethasone dipropionate (0.05%), diflorasone diacetate (0.05%), desoxymethasone, halobetasol propionate (0.05%) and fluocinonide (0.1%).

Class 2—potent, comprising 6 steroids: betamethasone dipropionate (0.05%), mometasone furoate (0.1%), diflorasone diacetate (0.05%), halcinonide (0.1%), fluocinonide (0.05%), desoxymethasone (O. 05%-0. 25%).

Class 3—upper mid-strength, comprising 3 steroids: fluticasone propionate (0.005%), fluocinonide (0.05%) and betamethasone valerate (0.12%).

Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).

Class 5—lower mid-strength, comprising 7 steroids: fluocinolone acetonide (0.01%), flurandrenolide (0.05%), fluticasone propionate (0.05%), prednicarbate (0.1%), desonide (0.05%), hydrocortisone (0.1%), hydrocortisone valerate (0.2%).

Class 6—mild, comprising only 3 steroids: alclomethasone dipropionate (0.05%), fluocinolone acetonide (0.01%), desonide (0.05%),

Class 7—least potent, comprising only one steroid: hydrocortisone (0.5%, 1%, 2%, 2.5%). The above topical drugs are marketed as lotions, creams, solutions, ointments, foam, sprays, gels.

Topical Tapinarof Combination Compositions

Provided herein are compositions, combinations, kits and articles of manufacture that include tapinarof in combination with tazarotene and optionally at least one corticosteroid of potency class 1-4, for treating plaque psoriasis. The compositions, combinations and articles of manufacture can be administered using a variety of routes such as topical application or transdermal application. The preferred route is the topical route and the preferred formulations are the cream and the lotion.

The optional corticosteroid in the compositions for the treatment of psoriasis may be superpotent (Class 1) or potent (Class 2). Alternatively, the steroid may be of lower potency (Class 3-4), thus minimizing the steroid side-effects, including the risk of pituitary suppression.

Therapeutically effective concentrations for treatment, prevention or amelioration of the symptoms manifested by plaque psoriasis of tapinarof and tazarotene is mixed with a suitable pharmaceutical carrier or vehicle for topical, transdermal or other routes.

Tapinarof and tazarotene in the combination compositions are included in an amount effective for treating, preventing or reducing the plaque psoriasis symptoms. The concentration of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and/or tazarotene in the marketed single drug currently administered for the treatment of plaque psoriasis. The dosage and regimen of administration may be determined by dose finding studies, as known in the art.

Exemplary dosages, strengths and concentrations of tapinarof in the tapinarof combination compositions administered topically, can be in the range of from about or at 0.1%, 0.25%, 0.5%, 1%, 2%, 2.5%,3%, 3.5%, 4%, 4.5% or 5%w/w. In another embodiment, the combination compositions administered topically, can be in the range of between 0.1 and 5%w/w, between 0.25 and 3% w/w, between 2.5% to 5% w/w. Typical strengths in the topical combination compositions of this invention are 0.25%, 0.5%, 1% or 2% w/w tapinarof. The frequency of administration can be determined empirically. Exemplary frequencies are once daily, twice daily, weekly, bi-weekly or monthly. Typical administration frequencies of the topical combination compositions of this invention are once daily and twice daily.

Dosage frequencies can be gradually attenuated over time and maintained at a steady dose suitable for long-term—6 months, 1 year, 5 years, 10 years or more, up to lifelong administration to control the symptoms of the plaque psoriasis. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, or any other formulation suitable for topical administration. The preferred compositions are the cream and the lotion.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients. The active agents are included in the carrier in an amount sufficient to exert a therapeutically useful effect i.e., amelioration of the symptoms of the plaque psoriasis, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include those suited for use include lotions, creams, solutions, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

Tapinarof Combination Compositions for the Treatment of Psoriasis

The optional at least one corticosteroid in the compositions of this invention for the treatment of plaque psoriasis may be superpotent (Class 1) or potent (Class 2). Alternatively, the steroid may be of lower potency, selected from upper mid-strength (Class 3), mid-strength (Class 4), thus minimizing the steroid side-effects, including the risk of pituitary suppression.

The superpotent (Class 1) or potent (Class 2) corticosteroids are selected from the following steroids (strengths are indicated in parentheses):

Class 1—superpotent, comprising 7 steroids: clobetasol propionate (0.05%), flurandrenolide (0.05%), betamethasone dipropionate (0.05%), diflorasone diacetate (0.05%), desoxymethasone, halobetasol propionate (0.05%) and fluocinonide (0.1%).

Class 2—potent, comprising 6 steroids: betamethasone dipropionate (0.05%), mometasone furoate (0.1%), diflorasone diacetate (0.05%), halcinonide (0.1%), fluocinonide (0.05%), desoxymethasone (O. 05%-0. 25%).

The lower potency steroids are selected from:

Class 3—upper mid-strength, comprising 3 steroids: fluticasone propionate (0.005%), fluocinonide (0.05%) and betamethasone valerate (0.12%).

Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).

Exemplary compositions of this invention are detailed in Examples 1-2.

Methods of Treatment

According to an aspect of the invention, there is provided a method of treatment of psoriasis by treatment of a subject in need thereof with a combination composition of tapinarof and tazarotene. In another embodiment, the psoriasis is plaque psoriasis, guttate psoriasis, inverse/flexural psoriasis, pustular psoriasis, erythrodermic psoriasis, sebo-psoriasis/seborrheic-like psoriasis, nail psoriasis, palmoplanar psoriasis or scalp psoriasis. In another embodiment, the psoriasis is plaque psoriasis. In another embodiment, the psoriasis is inverse/flexural.

In some embodiments, the effective amount is a therapeutically effective amount of tapinarof and tazarotene, namely an amount which will cure, treat, mitigate or prevent plaque psoriasis.

In some embodiments, co-administration of tapinarof and tazarotene exhibits an additive and/or synergistic effect while treating, preventing or alleviating plaque psoriasis.

In some other embodiments, the co-administration may be made either by administration of a single combination composition, or alternatively by separate administration of a first composition comprising tapinarof and a second composition comprising tazarotene.

### Regimen of Administration of the Topical Tapinarof-Tazarotene Combination Compositions

Therapeutically effective concentrations of tapinarof and tazarotene in the compositions of this invention for treatment, prevention or amelioration of the symptoms manifested by plaque psoriasis are determined by empirical methods known in the art.

The concentration of the active agents in the composition will depend on absorption, inactivation, excretion rates of the active compound, synergistic and/or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and tazarotene in the developed or marketed single drug currently being developed or used for the treatment of plaque psoriasis. The dosage and regimen of administration may be determined by dose finding studies, as known in the art.

Exemplary dosages, strengths and concentrations of tapinarof in the tapinarof combination compositions administered topically, can be in the range of from about or at 0.1%, 0.25%, 0.5%, 1%, 2%, 3%, 4% or 5% w/w. In another embodiment, the combination compositions administered topically, can be in the range of between 0.1 and 5%w/w, between 0.25 and 3% w/w, between 2.5% to 5% w/w.

Typical strengths in the topical combination compositions of this invention are 0.25%, 0.5%, 1% or 2% w/w tapinarof.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

### Treatment of Psoriasis with Tapinarof-Tazarotene-Corticosteroid Combinations

According to another, optional aspect of this invention, psoriasis is treated with tapinarof combinations with tazarotene and at least one corticosteroid of potency 1-4.

The corticosteroid used is of very high or high potency (Class 1 or 2, see above). Alternatively, the corticosteroid is selected from a lower group of potency (Class 3-4), which has milder side-effects and lower risk of pituitary suppression.

Due to the synergistic and/or additive effect with tapinarof, the corticosteroid can also be used at a lower strength (steroid-sparing) than the US-marketed topical drugs used for the treatment of psoriasis.

According to some embodiments, there is provided a method of treatment of psoriasis by topical administration to a subject in need thereof of a composition comprising a therapeutically effective amount of tapinarof, a therapeutically effective amount of tazarotene and a therapeutically effective amount of at least one corticosteroid selected from a superpotent (Class 1) corticosteroid a potent (Class 2) corticosteroid, an upper mid-strength (Class 3) corticosteroid, a mid-strength (Class 4) corticosteroid and a carrier suitable for topical administration.

In some other embodiments, the strength of the at least one corticosteroid in the compositions for the treatment of psoriasis is 25% w/w, 50% w/w or 75% w/w lower that the strength of the FDA-approved corticosteroids (see above FDA-approved strengths in parentheses).

Kits

Kits containing the combination compositions optionally including instructions for administration are provided. The combinations include, for example, the compositions as provided herein. Additionally, provided herein are kits containing the above-described combinations and optionally instructions for administration by topical, transdermal, or other routes, depending on the composition to be delivered.

The compositions provided herein can be packaged as articles of manufacture containing packaging material, a composition provided herein, and a label that indicates that the composition is for treating plaque psoriasis, and is formulated for topical delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

In some embodiments, there is provided a topical composition comprising from about 0.25% w/w to about 2.0% w/w tapinarof, from about 0.025% w/w to about 0.1% w/w tazarotene and a carrier suitable for topical administration.

In some other embodiments, there is provided a topical composition comprising from about 0.25% w/w to about 2.0% w/w tapinarof, from about 0.025% w/w to about 0.1% w/w tazarotene, from about 0.01% w/w to about 0.25% w/w at least one corticosteroid of potency class 1-4 and a carrier suitable for topical administration.

According to some embodiments, there is provided a dosage form comprising from about 0.25% w/w to about 2.0% w/w tapinarof, from about 0.025% w/w to about 0.1% w/w tazarotene, optionally from about 0.01% w/w to about 0.25% w/w at least one corticosteroid of potency class 1-4 and a carrier suitable for topical administration, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch and an applicator syringe.

According to some embodiments, there is provided a dosage form comprising from about 0.25% w/w to about 2.0% w/w tapinarof, from about 0.025% w/w to about 0.1% w/w tazarotene, optionally from about 0.01% w/w to about 0.25% w/w at least one corticosteroid of potency class 1-4 and a carrier suitable for topical administration, wherein the composition is formulated in a dosage form selected from a cream and a lotion.

In some embodiments, there is provided a method of treatment, prevention or alleviation of plaque psoriasis, by once daily or twice daily topical administration to a subject in need thereof of a therapeutically effective amount of a composition comprising from about 0.25% w/w to about 2.0% w/w tapinarof, from about 0.025% w/w to about 0.1% w/w tazarotene, optionally from about 0.01% w/w to about 0.25% w/w at least one corticosteroid of potency class 1-4 and a carrier suitable for topical administration, wherein the composition is formulated as a cream or as a lotion.

In some embodiments, there is provided a method of treatment, prevention or alleviation of plaque psoriasis, by once daily or twice daily topical administration to a subject in need thereof of a therapeutically effective amount of a composition comprising from about 0.25% w/w to about 2.0% w/w tapinarof, from about 0.025% w/w to about 0.1% w/w tazarotene and a carrier suitable for topical administration, wherein the composition is formulated as a cream or as a lotion.

In some embodiments, there is provided a method of treatment, prevention or alleviation of plaque psoriasis, by once daily or twice daily topical administration to a subject in need thereof of a therapeutically effective amount of a composition comprising from about 0.25% w/w to about 2.0% w/w tapinarof, from about 0.025% w/w to about 0.1% w/w tazarotene, from about 0.01% w/w to about 0.25% w/w at least one corticosteroid of potency class 1-4 and a carrier suitable for topical administration, wherein the composition is formulated as a cream or as a lotion.

According to some embodiments, there is provided a method of treatment of this invention, wherein tapinarof and tazarotene exhibit an additive or synergistic effect, thereby allowing to reduce the amounts of the active agents in the composition.

According to some other embodiments, there is provided a regimen of administration comprising the once daily or twice daily administration a patient in need thereof of a therapeutically effective dose of the corticosteroid-free composition comprising from about 0.25% w/w to about 2.0% w/w tapinarof, from about 0.025% w/w to about 0.1% w/w tazarotene and a carrier suitable for topical administration until complete remission.

In some embodiments, there is provided a regimen of administration comprising the once daily or twice daily administration a patient in need thereof of a dosage form selected from a cream and a lotion comprising from about 0.25% w/w to about 2.0% w/w tapinarof, from about 0.025% w/w to about 0.1% w/w tazarotene, optionally from about 0.01% w/w to about 0.25% w/w at least one corticosteroid of potency class 1-4 and a carrier suitable for topical administration,

In some other embodiments, there is provided a kit comprising one or more dosage forms comprising from about 0.25% w/w to about 2.0% w/w tapinarof, from about 0.025% w/w to about 0.1% w/w tazarotene, optionally from about 0.01% w/w to about 0.25% w/w at least one corticosteroid of potency class 1-4 and a carrier suitable for topical administration, wherein the composition is formulated in a dosage form selected from a cream and a lotion and instructions for use.

Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

### Example 1

### Preparation of a Tapinarof-Tazarotene Cream Composition

The topical tapinarof-tazarotene combination cream consists of:

0.25-2.0% w/w tapinarof,

0.025-0.1% w/w tazarotene,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl as an aqueous phase pH,

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, tazarotene, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-tazarotene combination cream in an aluminum tube or other delivery system.

### Example 2

### Preparation of a Tapinarof-Tazarotene-Corticosteroid Cream Composition

The topical tapinarof-tazarotene-corticosteroid combination cream consists of:

0.25-2.0% w/w tapinarof,

0.025-0.1% w/w tazarotene,

0.01-0.25% w/w corticosteroid of potency class 1-4,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl as an aqueous phase pH,

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, tazarotene, corticosteroid, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80, make up to 100% with purified water and adjust the pH to 6.0-7.0 with 0.1 M NaOH or HCl;

(5) add the aqueous phase to the oil phase under vacuum stirring, and cool to room temperature to obtain a cream;

(6) fill the tapinarof-tazarotene-corticosteroid combination cream in an aluminum tube or other delivery system.

### Example 3

### Preparation of Tapinarof, 1% Lotion

| Raw Material (compendial Name) | % w/w |
|---|---|
| Tapinarof 98% | 1.0 |
| Castor oil | 4.0 |
| Mineral oil light | 4.0 |
| Diethylene glycol monoethyl ether | 5.5 |
| Dimethyl Sulfoxide | 5.5 |
| Sorbitan Monooleate | 0.1 |
| Propylene glycol | 10.0 |
| Disodium Edetate (EDTA) | 0.1 |
| Carbomer Copolymer Type B | 0.4 |
| Pemulen ® TR-1 | |
| Carbomer Homopolymer Type A | 0.6 |
| Carbopol ® 981 | |
| Purified water | 64.00 |
| Benzoic Acid | 0.25 |
| BHT | 0.1 |
| Citric Acid | 0.1 |
| Sodium Citrate | 0.2 |
| Sodium hydroxide pellets | For pH adjustment |
| Purified water | q.s. to 100 |

Water phase

Into a glass beaker water and Benzoic Acid were added. The beaker was placed inside a hot water bath adjusted to 60° C. and the mixture was mixed with a magnetic stirrer until a clear solution free from particles was obtained. Then EDTA, Citric Acid and Sodium Citrate were added. The mixing was continued until a clear solution was obtained. The solution was cooled down to room temperature. Then, the pH was slowly adjusted to pH 6.0 with NaOH 20%.

Oil phase

In a separate glass beaker Mineral oil light, castor oil, span 80 and BHT were weighed. The beaker was placed inside a hot water bath adjusted to 60° C. and the mixture was mixed with a magnetic stirrer until a uniform solution was obtained. Then Carbopol®981 and Pemulen®TR-1 were slowly added and the mixing was continued until a homogenous mixture was obtained. The mixture was cooled down to room temperature.

Active phase

Into a separate glass beaker Propylene Glycol, Transcutol and DMSO were weighed. The mixture was mixed with a magnetic stirrer until a uniform homogenous solution was obtained. The beaker was covered with an aluminum foil and placed in a yellow light hood. Tapinarof was slowly added, and the mixing was continued for about 1 h until a clear solution free from particles was obtained.

The oil phase was slowly added to the water phase while homogenizing for about 5 minutes, until there were no lumps. Then, the active phase was slowly added to the Water +Oil phase while homogenizing for about 5 minutes.

Water was added for batch completion, and final pH was measured to conform it is around pH 5.

1. A topical composition comprising from about 0.25% w/w to about 2.0% w/w tapinarof, from about 0.025% w/w to about 0.1% w/w tazarotene and a carrier suitable for topical administration.

2. The composition of claim 1, further comprises from about 0.01% w/w to about 0.25% w/w at least one corticosteroid of potency class 1-4.

3. A dosage form comprising the composition of claim 1, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch and an applicator syringe.

4. A method of treatment, prevention or alleviation of plaque psoriasis, by once daily or twice daily topical administration to a subject in need thereof of a therapeutically effective amount of a composition of claim 1, wherein the composition is formulated as a cream or as a lotion.

5. (canceled)

6. (canceled)

7. The method of claim 4, wherein tapinarof and tazarotene exhibit an additive or synergistic effect, thereby allowing to reduce the amounts of the active agents in the composition.

8. A regimen of administration comprising the once daily or twice daily administration a patient in need thereof of a therapeutically effective dose of the corticosteroid-free composition of claim 1 until complete remission.

9. A regimen of administration comprising the once daily or twice daily administration a patient in need thereof of the dosage form of claim 3.

10. A kit comprising one or more dosage forms of claim 3 and instructions for use.

11. A dosage form comprising the composition of claim 2, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch and an applicator syringe.

12. A method of treatment, prevention or alleviation of plaque psoriasis, by once daily or twice daily topical administration to a subject in need thereof of a therapeutically effective amount of a composition of claim 2, wherein the composition is formulated as a cream or as a lotion.

13. The method of claim 12, wherein tapinarof and tazarotene exhibit an additive or synergistic effect, thereby allowing to reduce the amounts of the active agents in the composition.

14. A regimen of administration comprising the once daily or twice daily administration a patient in need thereof of a therapeutically effective dose of the corticosteroid-free composition of claim 2 until complete remission.

15. A regimen of administration comprising the once daily or twice daily administration a patient in need thereof of the dosage form of claim 11.

16. A kit comprising one or more dosage forms of claim 11 and instructions for use.
